(19) United States
(12) Patent Application Publication
(10) Pub. No.: US 2006/0041158 A1
(43) Pub. Date: Feb. 23, 2006

(54) Method for the production of an ester

(76) Inventors: Thomas Albers, Dusseldorf (DE); Neuss Michael, Koln (DE); Heiko Klaus, Dusseldorf (DE)

Correspondence Address: COGNIS CORPORATION;PATENT DEPARTMENT, 300 BROOKSIDE AVENUE, AMBLER, PA, 19002, US

(21) Appl. No.: 10/534,307

(22) PCT Filed: Oct. 30, 2003
(86) PCT No.: PCT/EP03/12044

§ 371 Date: May 9, 2005

(30) Foreign Application Priority Data
Nov. 8, 2002 (DE) 102 51 984.6

(51) Int. Cl. C07C 67/02

(52) U.S. Cl. 554/174

(57) ABSTRACT

A process for esterification of carbonyl compounds is provided. The process forms a mixture of a carbonyl compound and an inorganic phosphorous compound. An inorganic base can be added to the mixture. The mixture is filtered and an alcohol and a titanate catalyst are added to the filtrate. The mixture is esterified at an elevated temperature.

### FIELD OF THE INVENTION

This invention relates to a process for the production of esters, more particularly esters which are obtained by esterification of fatty acids or hydroxyfatty acids with alcohols or polyols and which are used, for example, as additives in cosmetic compositions or pharmaceutical preparations.

### PRIOR ART

A problem attending many esterification reactions and, above all, the production of fatty acid or hydroxyfatty acid esters is that the degree of esterification is often not high enough and the product obtained is yellowish to brownish in color. The degrees of esterification and discoloration are dependent inter alia on the esterification catalysts used. Thus, although acidic catalysts normally produce a light-colored product, the degree of esterification is unacceptable whereas basic catalysts produce dark-colored esters, but a high degree of esterification.

EP 0 000 424 B1, for example, describes the esterification of polymeric monocarboxylic acids, such as poly-(12-hydroxystearic acid), with alcohols, such as polyethylene glycol. Tetrabutyl titanate inter alia is mentioned as an esterification catalyst. However, the hydroxyfatty acid esters obtained are unsatisfactory in regard to color.

In order to reduce the discoloration of polymeric esters, catalysts consisting of a combination of several compounds have also been proposed. U.S. Pat. No. 6,080,834, U.S. Pat. No. 6,166,170 and U.S. 6,255,441 relate to catalyst compositions which may be used for the production of polyesters and especially polyalkylene terephthalates. The catalyst composition is obtained by reacting a titanium compound with a phosphorus compound in the presence of a complexing agent. Suitable titanium compounds are tetraalkyl orthotitanates. The phosphorus compound may be a phosphorus acid, such as hypophosphorous acid. However, if the described catalyst compositions are used for the esterification of fatty acids and especially hydroxyfatty acids with alcohols, such as polyols, the esters obtained are unsatisfactory both in regard to degree of esterification and in regard to color.

Accordingly, the problem addressed by the present invention was to provide a process for the production of an ester which would have a high degree of esterification coupled with minimal coloration. The process would be particularly suitable for the production of esterification products of fatty acids or hydroxyfatty acids and alcohols with the properties mentioned above.

### DESCRIPTION OF THE INVENTION

It has now surprisingly been found that esters of an organic carbonyl compound and an alcohol which combine a high degree of esterification with very little discoloration can be produced using a catalyst containing an inorganic phosphorus(I) compound and a titanate providing a certain sequence of process steps is observed.

Accordingly, the present invention relates to a process for the production of an ester of an organic carbonyl compound and an alcohol by carrying out an esterification reaction in the presence of a catalyst containing an inorganic phosphorus(I) compound and a titanate, characterized in that the organic carbonyl compound and the inorganic phosphorus(I) compound are first mixed together, the mixture obtained is filtered and the alcohol and the titanate are then added to the filtered mixture and the esterification reaction is carried out.

In contrast to the known processes where a catalyst component is initially prepared from an inorganic phosphorus(I) compound, titanate and complexing agent and is then added to the reaction mixture of acid and alcohol, the catalyst components are added separately from one another in various stages in the process according to the invention. The organic carbonyl compound is first pretreated with the inorganic phosphorus(I) compound and the mixture obtained is then filtered. Without wishing to be confined to any particular theory, the advantage of this procedure is presumably that the pretreatment with the phosphorus(I) compound and the subsequent filtration result in purification of the organic carbonyl compound. This clearly reduces the level of impurities which could lead to discoloration of the ester obtained in the subsequent reaction of the filtrate with the alcohol in the presence of the titanate. The presence of the titanate as an additional catalyst component in the actual esterification reaction ultimately leads to the high degrees of esterification required. Overall, therefore, products with very little color, but a high degree of esterification, are obtained in the process according to the invention.

Organic Carbonyl Compound

The process according to the invention is not confined to the production of certain esters. Basically, any esterifiable organic carbonyl compounds may be used, including carboxylic acids or carboxylic acid derivatives, more particularly carboxylic anhydrides, carboxylic acid halides, carboxylic acid salts, carboxylic acid amides or carboxylic acid esters. Whenever carboxylic acids in general are mentioned hereinafter as starting compounds for the esterification reaction, the above-mentioned derivatives of the corresponding acid are always understood to be included.

The process according to the invention is particularly suitable for the production of fatty acid esters with a fatty acid component containing 8 to 22 and preferably 12 to 18 carbon atoms. The carbonyl component may even be substituted. The process according to the invention may be used with particular advantage, for example, in the esterification of hydroxyfatty acids, such as those with 8 to 22 and preferably 12 to 18 carbon atoms in the fatty acid part. 12-Hydroxystearic acid is particularly suitable as starting compound for the esterification process according to the invention.

The monomeric fatty acids or hydroxyfatty acids described above may even be replaced by the corresponding polyfatty acids or polyhydroxyfatty acids, preferably those with a degree of self-condensation of 2 to 20 and more particularly 2 to 10. A preferred example of this group of organic carbonyl compounds is poly-12-hydroxystearic acid.

The esters of poly-12-hydroxystearic acid are used, for example, as emulsifiers in cosmetic preparations. They are present, for example, in the compositions which are marketed by Cognis Deutschland GmbH & Co. KG under the names of “Dehymuls® PGPH”, “Eumulgin® VL 75” (mixture with Coco Glucosides in a ratio by weight of 1:1) or “Dehymuls® SBL”. Polyol polyhydroxystearates are also described in EP 0 766 661 B1. These compounds can also be produced by the process according to the invention.

Alcohol

However, the process according to the invention is not confined to the alcohols described in EP 0 766 661 B1 as one of the starting components of the esterification reaction. Basically, any mono- and polyhydric alcohols may be used. The esterification reaction is preferably carried out using a polyol. Polyols containing 2 to 12 and more particularly 2 to 8 hydroxyl groups are preferred. Particularly preferred polyols are polyalkylene glycols, more especially polyethylene glycols, and also glycerol and polyglycerols.

The quantity of organic carbonyl compound and alcohol used in the process according to the invention is not particularly limited either and may be within the typical range for esterification reactions of these starting components. Besides complete esterification of the hydroxyl groups available, partial esterification thereof is also possible. A suitable molar ratio of organic carbonyl compound to alcohol is, for example, 1:1 to 30:1, more particularly 1:1 to 20:1 and, in a particularly preferred embodiment, 1:1 to 15:1.

Titanate

Basically, the titanate may be selected from any of the titanates already known as esterification catalysts. According to the invention, titanates capable of forming esters with the alcohol used are preferred. Preferred titanates are tetraalkyl orthotitanates, more particularly tetraisopropyl titanate and tetrabutyl titanate. Suitable quantities of titanate are, for example, 0.01 to 0.1% by weight, based on the total quantity of organic carbonyl compound, alcohol and catalyst in the reaction mixture. Too large a quantity of titanate can lead to unwanted discoloration of the ester obtained; too small a quantity can slow the esterification reaction.

Phosphorus(I) Compound)

Basically, the phosphorus(I) compound may also be selected from any of the phosphorus(I) compounds already known as esterification catalysts. According to the invention, preferred phosphorus(I) compounds are phosphorus(I) acid (i.e. phosphinic acid, hypophosphorous acid) or salts of phosphorus(I) acid (phosphinates, hypophosphites). Suitable quantities of the phosphorus(I) compound are 0.1 to 1% by weight, based on the total quantity of organic carbonyl compound, alcohol and catalyst in the reaction mixture.

In a first step of the process according to the invention, the organic carbonyl compounds and the inorganic phosphorus(I) compound are mixed for at least 20 minutes at a temperature of 20 to 220° C., preferably at a temperature of 60 to 180° C. and more particularly at a temperature of 80 to 120° C. The mixing times are temperature-dependent and are typically between 15 and 180 minutes. Mixing times of 30 to 60 minutes at temperatures of 80 to 120° C. are preferred.

Inorganic Base

Particularly good results in regard to the color of the esterification product obtained are achieved if an inorganic base is added to the mixture of organic carbonyl compound and inorganic phosphorus(I) compound before the filtration step. In a particularly preferred embodiment, the inorganic base is a basic salt, more particularly a carbonate such as, for example, sodium or potassium carbonate. The inorganic base is preferably added in a quantity sufficient to neutralize the phosphorus(I) compound substantially completely. Accordingly, the inorganic base is preferably added in an equivalent quantity to the phosphorus(I) compound used, although it may also be added in excess in relation thereto.

The advantage of adding an inorganic base presumably is, on the one hand, that, as a result of the pre-neutralization, the reaction mixture is not too acidic in the subsequent esterification step, so that a higher degree of esterification can be achieved. On the other hand, where an inorganic salt is added, the salt presumably acts as a filtration aid and thus improves the purifying effect of the filtration step.

Filtration Aid

Alternatively to or in addition to adding an inorganic salt, a filtration aid may be added to the mixture of organic carbonyl compound and inorganic phosphorus(I) compound before the filtration step. Basically, any mildly alkaline to mildly acidic known filtration aids, such as bleaching earths for example, are suitable. Suitable filtration aids are commercially available, for example, under the names of “Hyflow® Supercel” (Manville Corp.) or “Tonsil® Standard” (Südchemie). These filtration aids enhance the purifying effect of the filtration step and thus contribute towards reducing color in the end product.

The process according to the invention may be carried out either in the melt phase or in solution. From the cost and environmental perspectives, the process is preferably carried out in the melt. However, the reaction may also be carried out in a nonpolar, inert organic solvent. Particularly preferred solvents are those which form an azeotrope with the water of esterification formed, accompanied by a reduction in boiling point, such as toluene or xylene for example. The particular procedure selected in the esterification process according to the invention is primarily determined by the educts selected. The choice of suitable process parameters, such as the choice of suitable solvents and the choice of the reaction temperatures, is known in principle to the expert.

In order to minimize discoloration of the end product, the esterification temperature selected during the process according to the invention is as low as possible. Where it is carried out in the melt, the esterification reaction preferably takes place at a temperature below 240° C. and more particularly at a temperature of 180 to 220° C. Beyond a temperature of ca. 170° C., rapid discoloration is normally observed in the esterification step of the process according to the invention where phosphorus(I) acid or a salt thereof is used as the phosphorus(I) compound. This is presumably attributable to the decomposition of the phosphorus(I) acid which—via intermediate stages—forms phosphane and phosphoric acid which ultimately leads to bleaching of the reaction mixture.

Particularly good results are obtained if the reaction mixture is slowly heated to the maximum reaction temperature during the esterification step, so that water is continuously removed. This is particularly the case where hydroxyfatty acids are used as the organic carbonyl compound. An overly rapid increase in temperature can lead to the elimination of water from the hydroxyfatty acid and hence to the unwanted formation of unsaturated fatty acids in the reaction mixture.

Carrying out the reaction—either just the actual esterification or the reaction as a whole—in an inert gas atmosphere, such as nitrogen or argon, can lead to an improvement in the quality of the end product.

The invention is illustrated by the following Examples.

### EXAMPLES

### Example 1

### Synthesis of a polyethylene glycol polyhydroxystearate

5 g phosphorus(I) acid (50%) were added to 726.8 g (2.37 mol) 12-hydroxystearic acid, followed by stirring for 1 hour at 90° C. After addition of 8 g sodium carbonate and 5 g Hyflow® Supercel, the hot mixture was filtered. 273.2 g (0.18 mol) polyethylene glycol 1500 and 0.4 g Tyzor® TPT were added to the filtrate. The reaction mixture was slowly heated for 2 hours to 190° C. in an inert gas atmosphere (nitrogen) and esterified in vacuo for 18 hours during which the temperature was gradually increased to 210° C. and water was continuously removed. After cooling to ca. 100° C. and filtration, the product was obtained as filtrate.

The product had an acid value of 8, an iodine value of 2 and a Hazen color value of 100. The process according to the invention gives particularly light-colored and largely odorless products.

### Example 2

### Synthesis of a polyethylene glycol polyhydroxystearate

The procedure was as in Example 1 except that the 0.4 g Tyzor® TPT was replaced by 0.4 g Tyzor® TBT.

The product has an acid value of 8, an iodine value of 2 and a Hazen color value of 100.

### Comparison Example 1

### Synthesis of a polyethylene glycol polyhydroxystearate

672 g (2.19 mol) 12-hydroxystearic acid, 328.3 g (0.219 mol) polyethylene glycol 1500 and 0.2 g Ti(OBu)₄ were heated under nitrogen to 240° C. After the elimination of water had stopped, a vacuum was applied and the condensation reaction was continued until there was no further reduction in the acid value. After cooling to 100° C. and addition of 0.5% filter aid (Hyflow® Supercel), the product was filtered.

The product (filtrate) had an acid value of 9.5 and was dark brown in color. Neither the Hazen nor the Gardner color value could be determined.

### Comparison Example 2

### Synthesis of a polyethylene glycol polyhydroxystearate

754.3 g (2.46 mol) 12-hydroxystearic acid and 245.8 g (0.164 mol) polyethylene glycol 1500 were heated under nitrogen to 240° C. After the elimination of water had stopped, a vacuum was applied and the condensation reaction was continued until there was no further reduction in the acid value. After cooling to 120° C. and filtration after the addition of 0.5% filter aid (Hyflow® Supercel), the product was obtained as filtrate.

The product had an acid value of 23 and was dark brown in color. Neither the Hazen nor the Gardner color value could be determined.

### Comparison Example 3

### Synthesis of a polyethylene glycol polyhydroxystearate

506 g (1.65 mol) 12-hydroxystearic acid, 494 g (0.165 mol) polyethylene glycol 3000 and 5 g (0.038 mol) H₃PO₂ (50%) were heated under nitrogen to 240° C. After the elimination of water had stopped, a vacuum was applied and the condensation reaction was continued until there was no further reduction in the acid value. The catalyst was then neutralized with sodium carbonate. To this end, twice the theoretical quantity of Na₂CO₃ (anhydrous) (calculated from the acid value) was added to the product at 80° C. The salts were filtered off after the addition of 0.5% filter aid (Hyflow® Supercel). The product was obtained as filtrate.

The product had an acid value of 26 and was light brown in color. Neither the Hazen nor the Gardner color value could be determined.

Comparison Example 4

### Synthesis of a polyethylene glycol polyhydroxystearate

711 g (2.316 mol) 12-hydroxystearic acid were heated to 80° C., followed by the addition of 0.5 g H₃PO₂. After 30 minutes at 160° C., 289 g (0.193 mol) polyethylene glycol 1500 and, 30 minutes later, 0.4 g Ti(OBu)₄ were added under nitrogen and the whole was heated to 210° C. After the elimination of water had stopped, a vacuum was applied and the condensation reaction was continued at 240° C. until there was no further reduction in the acid value. After cooling to 120° C. and filtration after the addition of 0.5% filter aid (Hyflow® Supercel), the product was obtained as filtrate.

The product had an acid value of 11.1 and was dark brown in color. Neither the Hazen nor the Gardner color value could be determined.

### Example 3

### Synthesis of a polyglycerol polyhydroxystearate

5 g hypophosphorous acid were added to 893.6 g (2.91 mol) 12-hydroxystearic acid, followed by stirring for 1 hour at 90° C. After addition of 2 g sodium carbonate and 5 g filter aid (Hyflow® Supercel), the hot mixture was filtered.

106.4 g (0.64 mol) polyglycerol and 0.4 g tetrabutyl titanate were added to the filtrate. The reaction mixture was slowly heated for 2 hours to 190° C. in an inert gas atmosphere and, after the continuous removal of water, was condensed in vacuo for 18 hours while heating to 210° C. until there was no further reduction in the acid value. After cooling to ca. 100° C., the product was obtained as filtrate.

The product had an acid value of 0.8 and an iodine value of 5.3 and was light beige in color. The Gardner color value was 2.1 and the Hazen color value 299.

### APPENDIX

1) Hyflow® Supercel INCI: Kieselgur Manufacturer: Manville Corporation, USA
2) Tyzor® TBT INCI: Tetrabutyltitanat Manufacturer: E.I. du Pont de Nemours and Company, Wilmington, Del., USA
3) Tyzor® TPT INCI: Tetraisopropyltitanat Manufacturer: E.I. du Pont de Nemours and Company, Wilmington, Del., USA

1-15. (canceled)

16. A process for the production of esters of organic carbonyl compounds with alcohols by carrying out an esterification reaction in the presence of a catalyst containing an inorganic phosphorus(I) compound and a titanate, which comprises:
(1) mixing the organic carbonyl compound and the inorganic phosphorus(I) compound to form a mixture;
(2) filtering the mixture;
(3) adding the alcohol and the titanate to the filtered mixture to form an alcohol-carbonyl mixture; and
(4) esterifying the carbonyl compound in the alcohol-carbonyl compound mixture.

17. The process as claimed in claim 16, wherein, the carbonyl compound comprises a fatty acid containing 8 to 22 carbon atoms.

18. The process as claimed in claim 15, wherein, the carbonyl compound comprises a hydroxyfatty acid containing 8 to 22 carbon atoms.

19. The process as claimed in claim 16, wherein, the organic carbonyl compound comprises at least one fatty acid selected from the group consisting of polyfatty acids and polyhydroxyfatty acids with a degree of self-condensation of 2 to 20.

20. The process as claimed in claim 16, wherein, the alcohol comprises a polyol.

21. The process as claimed in claim 16, wherein, the titanate comprises a titanate which is capable of forming esters with the alcohol.

22. The process as claimed in claim 21, wherein, the titanate is added in a quantity of 0.01 to 0.1% by weight, based on the total quantity of organic carbonyl compound, alcohol and catalyst.

23. The process as claimed in claim 16, wherein, a phosphorus(I) acid or salt thereof is used as the phosphorus(I) compound.

24. The process as claimed in claim 23, wherein, the phosphorus(I) compound is present in a quantity of 0.1 to 1% by weight, based on the total quantity of organic carbonyl compound, alcohol and catalyst.

25. The process as claimed in claim 16, wherein, before the filtration step (2), an inorganic base is added to the mixture containing the organic carbonyl compound and the inorganic phosphorus(I) compound.

26. The process as claimed in claim 25, wherein, the inorganic base is added in a quantity sufficient to neutralize the phosphorus(I) compound substantially completely.

27. The process as claimed in claim 16, wherein, in step (1) of the process, the organic carbonyl compound and the inorganic phosphorus(I) compound are mixed for at least 20 minutes at a temperature of from 20° C. to 220° C.

28. The process as claimed in claim 16, wherein, the esterification reaction is carried out in a melt or in a nonpolar, inert organic solvent.

29. The process as claimed in claim 16, wherein, the esterification reaction is carried out in a melt at a temperature below 240° C.

30. The process as claimed in claim 16, wherein, at least the esterification reaction is carried out in an inert gas atmosphere.

31. The process as claimed in claim 16, wherein, the alcohol comprises at least one member selected from the group consisting of polyalkylene glycol, glycerol and polyglycerol.

32. The process as claimed in claim 16, wherein, the titanate comprises a tetraalkyl orthotitanate.

33. The process of claim 32, wherein, the titanate comprises at least one member selected from the group consisting of tetraisopropyl titanate and tetrabutyl titanate.

34. The process of claim 25, wherein, the inorganic base comprises a carbonate.

35. The process of claim 17, wherein, the fatty acid contains from 12 to 18 carbon atoms.
